(19) United States
(12) Patent Application Publication
(10) Pub. No.: US 2010/0191095 A1
(43) Pub. Date: Jul. 29, 2010

(54) PROCESS, DEVICE AND SYSTEM FOR REDUCING THE ARTIFACTS THAT AFFECT ELECTROPHYSIOLOGICAL SIGNALS AND THAT ARE DUE TO ELECTROMAGNETIC FIELDS

(75) Inventors: Jacques Felblinger, Mereville (FR); Michel Kraemer, Durrenbach (FR); Jean-Philippe Blonde, Souffelweyersheim (FR); Roger Abacherli, Baar (CH); Vincent Frick, Strasbourg (FR); Johann-Jakob Schmid, Rifferswil (CH)

Correspondence Address: YOUNG & THOMPSON, 209 Madison Street, Suite 500, Alexandria, VA, 22314, US

(73) Assignees: SCHILLER MEDICAL, WISSEMBOURG (FR); UNIVERSITE HENRI POINCARE-NANCY I, NANCY (FR); UNIVERSITE DE STRASBOURG, STRASBOURG (FR); CENTRE HOSPITALIER ET UNIVERSITAIRE DE NANCY, NANCY (FR)

(21) Appl. No.: 12/597,798

(22) PCT Filed: Apr. 25, 2008
(86) PCT No.: PCT/FR2008/050751

§ 371 Date: Apr. 1, 2010

(30) Foreign Application Priority Data
Apr. 27, 2007 (FR) 0754747

(51) Int. Cl. A61B 5/055

(52) U.S. Cl. 600/411

(57) ABSTRACT

A method for collecting and a device for sensing at least one electrophysiological signal, as well as to a system including at least one such device. The device for sensing at least one electrophysiological signal from a living subject subjected to at least one electromagnetic field, includes elements, such as electrodes, which are placed on the subject for the physical acquisition of at least one electrophysiological signal and at least elements for the pre-processing of the at least one sampled electrophysiological signal. The sensor device (1) is characterised in that it also includes additional elements (7, 8) for measuring or determining at least one characteristic of the electromagnetic environment at or close to the electrophysiological signal acquisition point(s) or zone(s) on the subject.

This invention relates to a process, a sensor device, and a system whose objective is the collection of at least one electrophysiological signal for its display, its printing, its analysis, and its interpretation within the framework of the monitoring of the patient in whom it is collected and for the activation of secondary functions.

These secondary functions can be the prospective or retrospective synchronization of an imager (for example, Magnetic Resonance Imagery, Positron Emission Tomography, X-Ray Tomodensitometry, Scintigraphy, Echography).

An example of a device and a system that satisfy this objective is the monitor of the applicant that is known under the designation Maglife C, which collects the electrocardiogram in an MRI environment.

Magnetic resonance imagery allows non-invasive examinations in routine clinical practice. This technique uses a high-value magnet, primarily in the form of a tunnel, in which the patient lies down. To produce the images, gradients of magnetic fields and radiofrequency pulses are used during sequences that are selected based on desired images.

The electrophysiological signals that are generally involved in the scope of this invention are the electrocardiogram (EKG), the electroencephalogram (EEG), the electromyogram (EMG), and the electro-occulogram (EOG). These signals can be used during the MRI examinations either for a surveillance of the patient (“monitoring”) or for the synchronization of MRI sequences or else for research purposes.

These electrical signals are unfortunately disrupted by the MRI environment during the generation of images. On this subject, it is possible to cite, for example, the following publications:

J. Felblinger, C. Lehmann, C. Boesch, “Electrocardiogram Acquisition during MR Examinations for Patient Monitoring and Sequence Triggering,” Magnetic Resonance in Medicine, 32, 523-529 (1994);

J. Felblinger, J. Debatin, C. Boesch, R. Gruetter, G. McKinnon, “Synchronization Device for EKG-Gated Echo Planar Imaging of the Human Heart,” Radiology, 197, 311-313 (1995);

J. Felblinger, R. M. Mueri, C. Ozdoba, G. Schroth, C. W. Hess, C. Boesch, “Recording of Eye Movements for Stimulus Control during fMRI by Means of Electro-oculographic Methods,” Magnetic Resonance in Medicine, 36, 410-414 (1996);

R. M. Mueri, J. Felblinger, K. M. Roesler, B. Jung, C. W. Hess, C. Boesch, “Recording of Electrical Brain Activity in a Magnetic Resonance Environment: Distorting Effects of Static Magnetic Field,” Magnetic Resonance in Medicine, 39, 18-22 (1998).

The use of the electrocardiogram for the prospective or retrospective synchronization of the imager does not necessarily require a complete elimination of the interference in the signal. The synchronization signal can be obtained from the combination and the processing of several electrocardiological signals that are collected simultaneously (US-2006/173249).

The document U.S. Pat. No. 4,991,580 proposes a method for detecting the QRS wave (starting from which the synchronization process is performed) that is based on the research and the recording of a form of this wave before the MRI examination, which causes this interference.

There are also methods for synchronization of the taking of images with cardiac activity that are not based on the collection of electrocardiograms. They are based on the use of images that are made by the imager itself to obtain information on the cardiac activity with which the image-taking is to be synchronized. These techniques are accessible only in recent and dedicated MRI imagers.

Regarding the collection of electrocardiograms for the purpose of monitoring patients, and from which maximum undesirable interference therefore is eliminated, various solutions have been proposed in recent years.

Very simple methods consist in braiding the acquisition wires (Wendt, 1988) and applying variable filters based on the presence or absence of magnetic field gradient emissions (Rokey, 1988).

These methods have become inadequate because of the evolution of MRI technology, in particular with the increase in amplitude, switching speed, and the frequency of repetition of the field gradients.

A first technological jump (U.S. Pat. No. 5,782,241) consisted in placing all amplification electronics close to the measuring point and using an optical or RF transmission to transmit the signal beyond the magnet of the imager. This solution made possible significant progress for the reduction of interference in the electrophysiological signals and presents a particularly suitable method for transmission, since an optical connection is insensitive to electromagnetic fields and cannot involve risks of heating and burns.

A range of solutions based on learning the effect of magnetic field gradients on the electrophysical signals emerged.

Laudon (1998) uses a conductive loop that is close to the measurement location of the EKG to collect signals that are identical to the interference that is superposed on the electrocardiological signal so as to be able to remove it (subtraction). This approach is not entirely satisfactory because it has been shown that the largest portion of the artifacts is generated in the tissues, which the conductive loop cannot measure (Felblinger, 1994).

Via the patent U.S. Pat. No. 6,873,869 (or EP 1 050 270), a process was proposed that consists in simultaneously measuring two electrophysiological signals, one using two electrodes that are placed on the thorax, approximately on both sides of the heart, and the other using two electrodes that are placed symmetrically to the preceding ones relative to the median line of the thorax, on the same horizontal line. The first set of electrodes primarily measures the electrophysiological EKG signal with the interference, and the second primarily measures the interference. By carrying out a subtraction of these two signals, it was possible to reduce the interference in numerous cases.

However, the two measurements are not made at the same location; hence, taking into account interference at a zone that is separate from the measurement zone results. In addition, the second set of electrodes does not measure only the interference but also a portion of the useful signal, and consequently its subtraction from the signal that is provided by the first set of electrodes does not make it possible to end reliably in a non-reduced and non-disrupted electrophysiological signal.

An enhancement of this process, which is based on the estimation of the interference only starting from electrophysiological signals, can be obtained by the use of more sophisticated signal processing methods (for example, by adaptive filtering, analysis of primary or independent components, . . . ).

Allen (2000) proposes a method that is based on the elimination of the mean artifact that is calculated for a learning sequence. This method is based on the fact that the artifact is identical during the entire sequence, which is generally the case for the functional MRI but cannot be generalized to the MRI.

Sijbers (1999) uses the data from the applied MRI sequence. This solution is possible only in research systems for which the MRI sequences that are used are totally known.

Other different techniques for signal processing have been developed. They are all based on the knowledge of currents for generation of the magnetic field gradients. Felblinger (1999) proposes calculating the pulse response of the system with magnetic field gradients in three directions, by using a specific sequence. With these pulse responses, it is then possible to correct in real time the artifacts that are produced during any sequence.

A generalized approach was proposed by Odille (2006) by using a more general deconvolution method to find the pulse responses.

Still by using the currents for generating field gradients, Abächerli (2005) proposes an adaptive approach for finding the pulse response during any sequence. An adaptive method is also proposed by Kreger (U.S. Pat. No. 6,675,036).

These last methods are based on the connection of the signal processing system to the control electronics of the MRI gradients. By knowing the source of interference by the measurement of the currents that are injected in the gradient coils, it is possible to determine the disruptions that are generated at each moment.

However, these methods present certain difficulties and limitations of practical implementation. Thus, the connection to the control electronics of the gradients poses different problems, because it is necessary to make a direct connection between the signal processing module and the MRI cabinet, which cannot always be easily produced.

Furthermore, because of the undesirable Foucault currents that are induced, the currents of the gradient coils do not correspond exactly to the local magnetic fields that are produced and that can be measured, and therefore no longer correspond to the disruptions induced by these fields. Actually, the currents in the coils take into account the compensation of these Foucault currents so as to obtain the desired field at any point of the volume that is called “Field of View” (champ de vue), which is the zone that is possible for the imagery.

In addition, if the sensor is found beyond this space in which the field varies linearly (FOV or Field of View—champ de vue), the value of the current of the gradients does not correspond to the real field that is close to the sensor.

This invention has as its primary object to propose a solution that makes it possible in particular to overcome the above-mentioned difficulties and limitations by taking into consideration the local real disruptive factor, and neither its generation, nor its effects.

For this purpose, the invention has as its object a process for collecting—with a sensor device—at least one physiological signal of a living subject who is subjected to at least one electromagnetic field, in particular subjected to an electromagnetic environment such as the one existing in an investigative device that uses magnetic resonance, consisting in implementing a physical acquisition of at least one electrophysiological signal at the level of or on the subject, and at least one pre-processing operation of said at least one electrophysiological signal that is detected; this is done via corresponding means, a process that is characterized in that it also consists in producing, using additional means, a measurement of at least one characteristic of the electromagnetic environment at or in the immediate proximity of the point or points or zones for acquisition of the electrophysiological signal(s) on the subject and in correcting the—or each—electrophysiological signal SP that is detected and pre-processed, based on said at least one measuring signal SM, optionally pre-processed, of at least one characteristic of the electromagnetic environment of the point or points or zones for acquisition of the above-mentioned signal SP.

The principle on which this invention is based consequently consists in—unlike the known processes that are presented above—measuring, preferably in a continuous manner, at least one characteristic of the magnetic field and the variations of the latter at the very location where the electrophysiological signal is collected, disrupted by the variations of said magnetic field.

The pre-processing of said at least one detected signal can comprise in particular one or more of the following operations: amplification, filtering, selection of the processing channel or channels.

The characteristic of the electromagnetic environment that is measured directly, at the level of or in the immediate proximity of the—or of each—acquisition point of the electrophysiological signal or signals, can be the amplitude of the local magnetic field vector in one or more directions.

One variant consists in measuring directly, at or in the immediate proximity of the—or of each—acquisition point of the electrophysiological signal or signals, the value of the magnetic field gradient or gradients that is/are present, in one or more directions, whereby these field gradients are the primary disrupters and artifact generators for the measurements of electrophysiological signals in the MRI environment.

According to a first variant embodiment of the invention, the process can also consist in carrying out, with suitable means, a modulation, an adaptation to the transmission mode and an emission to a unit for control and remote processing, optionally combined with means for printing and/or display of said at least one pre-processed electrophysiological signal and said at least one signal for measuring at least one characteristic of the electromagnetic environment of the point or points or zones for acquisition of the above-mentioned signal, optionally also pre-processed.

According to a second variant embodiment of the invention, the process can consist in carrying out the corrective processing of the—or of each—electrophysiological signal SP based on said at least one measuring signal SM using a suitable means that is integrated with the sensor device and then in carrying out, with suitable means, a modulation, an adaptation to the mode of transmission and an emission to a unit for control and remote processing, optionally combined with means for printing and/or display of said at least one corrected electrophysiological signal.

The invention also relates to a sensor device of at least one physiological signal of a living subject who is subjected to at least one electromagnetic field, in particular subjected to an electromagnetic environment as it exists in an investigative device that uses magnetic resonance, comprising means for physical acquisition of at least one electrophysiological signal on the subject, such as electrodes, and at least means for pre-processing of said at least one detected electrophysiological signal, a sensor device that is characterized in that it also comprises additional means for the measurement of at least one characteristic of the electromagnetic environment at or close to the point or points or zones for acquisition of the electrophysiological signal or signals on the subject.

The above-mentioned additional means advantageously comprise at least one magnetic field sensor that provides a measuring signal based on the amplitude of the magnetic field vector at the level of said sensor and at least one signal pre-processing means delivered by the—or each—sensor.

These additional means are advantageously housed in the housing that contains the components of the sensor device, but they can also be directly combined with electrodes or be housed within a separate housing.

According to a constructive variant, the sensor device comprises at least one pair of magnetic field sensors that are arranged in a spaced manner in a determined direction, so as to provide the value of at least one field gradient in one direction.

According to a practical embodiment of the invention, the additional means comprise at least one magnetic field sensor in the form of a Hall effect sensor to measure the amplitude of the local magnetic field vector, or, according to a variant, at least one pair of Hall effect sensors that are arranged in a spaced manner in a direction that is determined for measuring the value of at least one magnetic field gradient in one direction.

Consistent with a first variant embodiment, the electrophysiological and magnetic measuring signals are transmitted separately from the sensor device, and the corrective processing of the first signals based on the second signals is carried out outside of the sensor device, for example by a processing unit that is located at a distance from said sensor device, in or beyond the chamber that contains the magnetic resonance device. In this case, the sensor device also comprises means for modulation, for adaptation to the mode of transmission and of emission for said at least one pre-processed electrophysiological signal and for said at least one signal for measuring at least one characteristic of the electromagnetic environment of the point or points or zones for acquisition of the above-mentioned signal, optionally also pre-processed.

Consistent with a second variant embodiment, the correction of the electrophysiological signal or signals is carried out at the sensor device itself. In this case, the latter also comprises, on the one hand, a means for correcting the—or each—detected and pre-processed electrophysiological signal based on said at least one measuring signal, optionally pre-processed, of at least one characteristic of the electromagnetic environment of the point or points or zones for acquisition of the above-mentioned signal, and, on the other hand, a means for modulation, for adaptation to the mode of transmission and emission for said at least one corrected electrophysiological signal.

The sensor device is adapted to make possible in an advantageous manner the implementation of the variants of the process described above.

So as to reduce in particular to a maximum the apparent surface of the sensor device that is subjected to the influence of the electromagnetic environment and to its variations, provision can be made that the means for pre-processing said at least one detected electrophysiological signal and the additional means, as well as optionally the means for modulation and adaptation to the mode of emission and the means for correction, are produced in the form of integrated microelectronic components (so as to avoid as much as possible any disruption of the surrounding electromagnetic field), preferably mounted or formed on a printed circuit or a similar substrate, preferably cast or mounted in a suitable protective housing.

The invention also has as its object a system for acquisition of at least one electrophysiological signal of a living subject who is subjected to an examination by an NMR device, in particular an MRI (magnetic resonance imaging) examination, whereby this system comprises in particular a unit for control and for processing and at least one sensor device that is connected to said unit, whereby said at least one sensor device is positioned on the subject and the processing unit is located at a distance, a system that is characterized in that the—or each—sensor device is a sensor device as mentioned above and as described in more detail below.

This system will make possible in particular the implementation of the process that is described above.

The invention will be better understood using the description below, which relates to preferred embodiments, provided by way of nonlimiting examples and explained with reference to the accompanying diagrammatic drawings, in which:

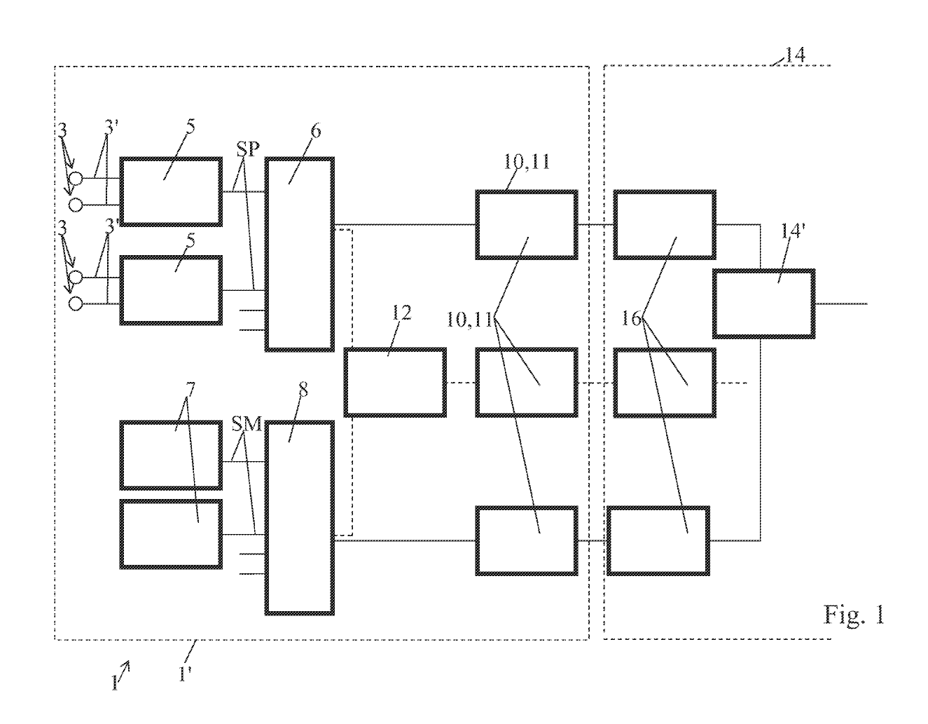

FIG. 1 is a simplified schematic diagram of a sensor device according to the invention;

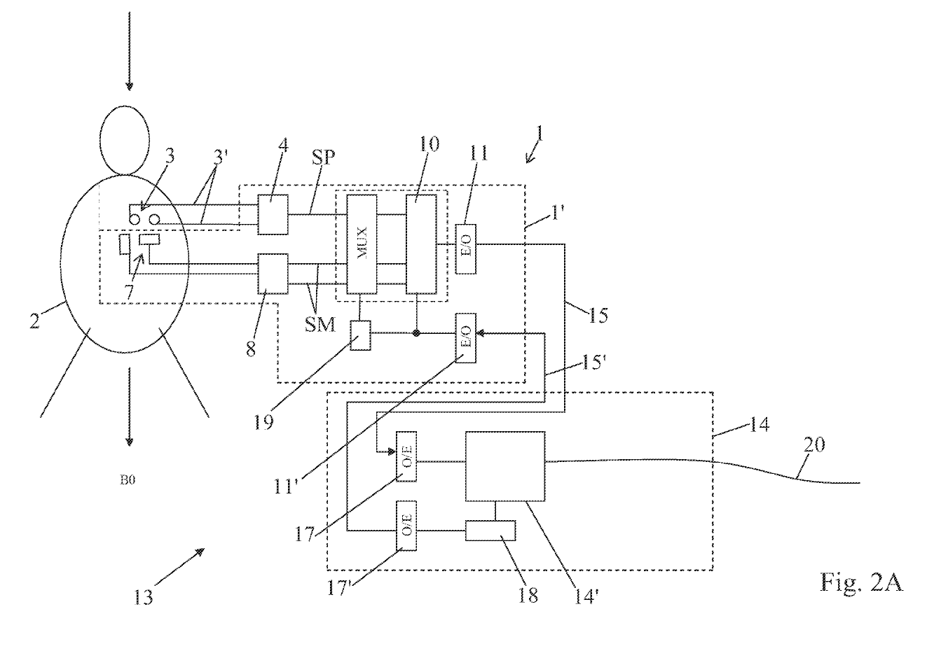

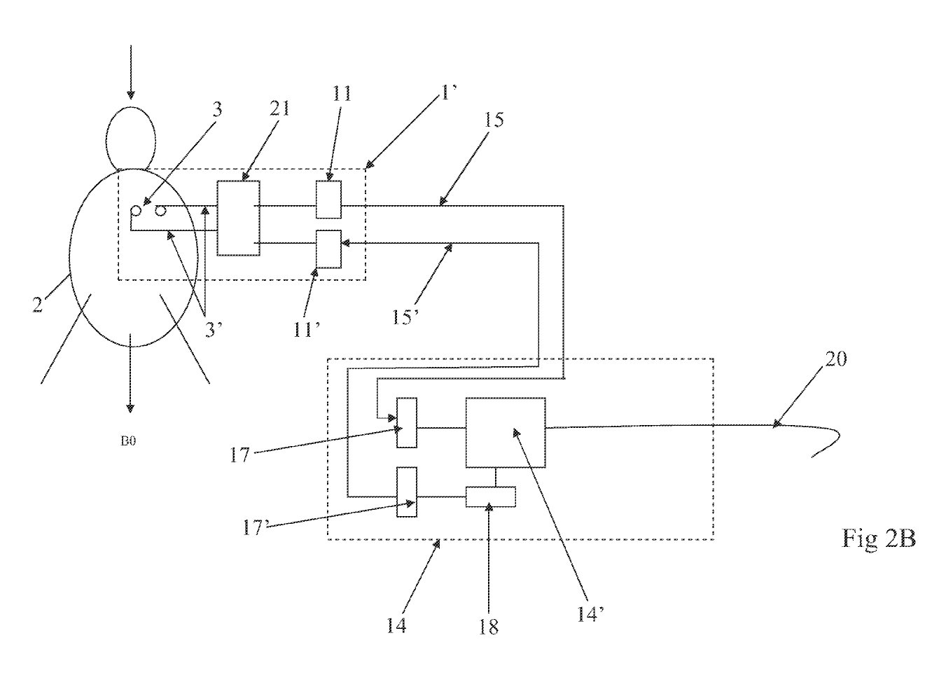

FIG. 2A is a schematic diagram of an acquisition system according to the invention, comprising a sensor device;

FIG. 2B is a schematic diagram of an acquisition system according to the invention that constitutes an equivalent variant embodiment, but more integrated, of the one that is shown in FIG. 2A, and

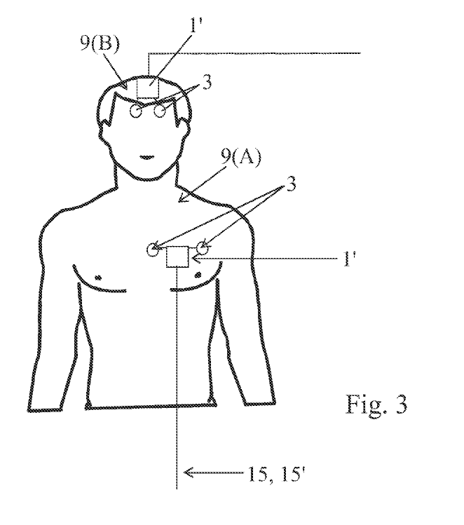

FIG. 3 is a partial diagrammatic top view of a reclining human subject illustrating possible positions for the electrodes of the sensor device according to the invention to detect electrocardiogram signals 9(A) and to detect an electroencephalographic signal 9(B).

As FIGS. 1 and 2 of the accompanying drawings show, the acquisition system 13 primarily consists of two sub-assemblies, namely:

One or more sensor devices 1 (a single one is shown) that make(s) possible the collection of at least one electrophysiological signal SP (for example: EEG, EKG, . . . ) from at least one measuring signal SM of the local magnetic field. In the example of FIG. 2, the patient is placed in the tunnel of the imager, and the measured field B0 is parallel to the large shaft of this tunnel.

A unit 14 for processing and for control placed at a distance from the patient, optionally outside of the insulated chamber that contains the magnetic resonance device.

The different constituent elements and the possible structures and methods of operation can be described in a general manner in connection with FIG. 1.

As the latter shows, the sensor device 1 comprises, from the functional standpoint, essentially two chains for acquisition and for at least pre-processing of signals, namely:

A chain that is dedicated to the electrophysiological signals SP that are detected on the subject 2, and

A chain that is dedicated to the signals SM that are representative of the local magnetic environment, preferably directly at measuring points 9 of the electrophysiological signals SP.

The sensors 7 that measure the local magnetic characteristics are preferably integrated in the protective housing 1′ of the sensor device 1, whereas the electrodes 3 that detect the signals SP are generally connected to the sensor device 1 by conductors 3′ of short length, and even conductors mounted on the underside of said housing 1′.

The chain that is dedicated to the electrophysiological signals SP can comprise several channels (two channels are shown in FIG. 1), and each channel for collecting an electrophysiological signal consists of at least two electrodes 3 that are connected to a corresponding signal amplifier 5 by means of short conductive cables 3′. A module 6 for conditioning signals SP, forming pre-processing means with the amplifier 5, ensures the filtering and the selection of acquisition channels. The selection of the bypasses can also be carried out upstream from the amplifier 5.

Several chains for acquisition of different electrophysiological signals SP can be combined in the sensor device 1.

Simultaneously and in parallel, a measurement of one or more characteristics of the local magnetic field is made in said sensor device 1. This measurement is advantageously carried out by using the Hall effect at one or more sensors 7, but other techniques could be used. The number of measuring points, and therefore of sensors 7, of the magnetic field can be easily increased due to the use of the microelectronic technology. By measuring the local magnetic field at two, four or six points, it is possible to determine, at a pre-processing module 8 that receives the signals that are obtained from sensors 7, the field gradients in one or more, and even all, directions, and this directly at the measuring point or points 9 of the electrophysiological signal SP.

For this purpose, it is advantageous to use sensors 7 that are assigned to the measurement of a given gradient with a maximum common spacing (for example, in the proximity of the edges that are opposite to the housing 1′ of the sensor device 1).

So as to allow a precise taking into account of the disruptions, each sensor 7 can consist of a Hall sensor in the form of a microelectronic circuit of a standard semi-conductor CMOS, preferably of the type that has a surface area of several mm² (for example, 1 to 15 mm², preferably from about 1 to 10 mm²), and a measurement precision on the order of 10 to 100 microtesla.

Thus, it is possible to measure fields and very slight variations of fields (typically on the order of milli-tesla) relative to the primary resident field (on the order of 0.5 T to 3 T).

In addition, the selection of such a technical solution based on semiconductor sensors instead of the conventional devices that consist of conductive loops through which the magnetic field passes avoids interfering with the medical images and/or causing heating in case of contact with the skin of the patient.

For the consecutive and correlative processing of the measuring signals SP and SM that are provided respectively by the electrodes 3 and the sensors 7, two alternative solutions can be considered:

Either the correction of the magnetic interference that affects the electrophysiological signals SP is done directly in the sensor device 1 by a processing circuit 12 that forms a means of correction (shown in dotted lines in FIG. 1),

Or all of the electrophysiological signals SP and signals for measuring magnetic fields SM are transmitted toward the outside of the sensor device 1 after pre-processing and conditioning and processing of the magnetic interference is carried out outside of the sensor device 1, in a suitable module 14′ of the unit for control and processing.

In the two above-mentioned variants, a modulation and an adaptation of the signals in the emission mode are produced in a suitable module 10, 11, and then the signals are transmitted to the system 14 by any mode of transmission that is compatible with a magnetically-disrupted MRI-type environment.

Preferably, the transmission of the pre-processed or pre-processed and corrected electrophysiological signal or signals SP, and if necessary, the signal or signals SM for measuring the magnetic field, or magnetic field gradients, is carried out according to a serial transmission mode that is selected from the group that is formed by the transmission by optical fibers, the transmission by radiofrequency waves, and the transmission by shielded electrical conductors.

At the level of the reception of these signals, means for conversion and for demodulation, for example in the form of adapted modules 16 of the outside unit 14, process the signals that are received before their exploitation.

It should be noted that the sensor device 1 that comprises the above-mentioned means 4 to 12 is designed so as to have the lowest possible consumption, thus allowing an independent supply. One of the basic principles guiding its design can be the use of a standard microelectronic technology.

In connection to FIG. 2A of the accompanying drawings, a variant embodiment of the overall system according to the invention, more particularly in connection to the acquisition of EKG signals, is described below by way of example.

In the form of block diagrams, this figure shows a system 13 for acquisition of at least one electrophysiological signal SP of a living subject 2 who is subjected to an examination by an MRI examination. This system 13 comprises in particular a unit 14 for control and processing and at least one sensor device 1 that is connected to said unit, whereby said at least one sensor device 1 is positioned on the subject 2 and the processing unit is located at a distance.

The—or each—sensor device 1 (a single one is shown in FIG. 2A), supplied by, for example, a cell, a battery or light energy, makes possible the collection of the EKG of a subject 2 using two electrodes 3 that are connected to the housing 1′ by means of two conductors 3′, preferably the shortest possible.

A stage or module 4, comprising at least one instrumentation amplifier and a filtering device, together forming the pre-processing means, delivers the EKG signal (SP) with the desired gain, dynamics and bandwidth.

One or two Hall sensors 7, perpendicular to one another, which measure the amplitude of the magnetic field vector that passes through them, are placed in the sensor device 1.

The stage or module 8 produces a shaping of the signals SM that are obtained from the Hall sensors 7 with the desired gain, dynamics and bandwidth (pre-processing means).

The two or three signals that are measured are, in modules that are common to two acquisition chains (10, MUX, 11), multiplexed (at MUX), then modulated (at 10) (for example, modulation of pulse width) so as to be able to be transmitted in series on a single optical fiber 15 after electronic/optical E/O transformation (at 11).

The optical fiber 15 connects the sensor device 1 and the processing unit 14, which can be placed at a distance.

The optical signals are reproduced in electronic form in an O/E conversion module 17 and transmitted to a processing module 14′ to be demultiplexed, demodulated and digitized there. The processing for eliminating interference is carried out starting from these digital data.

The synchronization of the serial connection 15, 15′ between the—or each—sensor device 1 and the processing unit 14 is ensured by a clock 18 that is transmitted by the optical fiber 15′ after electrical/optical E/O transformation in a module 17′ that is adapted and used after optical/electric O/E transformation (at 11′) for the control of the modulator 10 and the multiplexer MUX, via the meter 19.

Thus, the unit for control and processing 14 is connected to the—or to each—sensor device 1 via a bidirectional serial connection 15, 15′ that allows the transmission 15 of the—or of each—physiological signal SP that is collected by the sensor device(s) 1, and, if necessary, said at least one measuring signal SM of at least one characteristic of the magnetic environment at the measuring point(s) 9 of the sensor device 1 that is involved, to said unit 14 and the transmission 15′ of a clock signal from this unit 14 to said sensor device(s) 1, in particular for the purpose of modulation and multiplexing signals that are collected by the—or each—sensor device 1.

Provision can be made that the unit for control and processing 14 comprises at least one means for edition of the—or of each—physiological signal SP that is collected, for example a display means and/or a printing means, as well as a storage means.

It can also be provided that the unit for control and processing provides to the MRI device a sequencing signal that is extracted from the physiological signal SP that is collected; this is done by a suitable transmission connection 20.

The processing for correcting the magnetic interference can be done according to algorithmic techniques that are similar to those that are already known (see the publications cited above). Other methods are possible, however.

As FIG. 2B of the accompanying drawings shows by way of advantageous variant, provision can be made that the sensors 7 be integrated with the modules 4 and 8 for pre-processing signals SP and SM, as well as with the multiplexing circuits MUX and modulation circuits 10, in the same microelectronic circuit 21 that is mounted in a housing 1′ that is connected to or that carries the electrodes 3, or else integrated in the latter.

This circuit 21 can also integrate, if necessary, the means 12 that carries out the correction of the signal or signals SP based on the signal or signals SM (in software, material, or material and software form).

Such an integration, and the miniaturization that results therefrom, makes it possible to achieve a very reduced volume for the housing 1′, which reduces the impact of field gradients on the measurement circuits and therefore on the EKG that is collected, reduces the formation of artifacts on the images that are taken under the sensor because of its presence and therefore makes it possible to place this housing 1′ very close to the electrodes 3 even in the case of cardiac imagery. This allows the use of very short cables 3′ and therefore contributes to reducing even more the impact of field gradients on the EKG. This reduction in volume, by making it possible to place sensors 7 very close to the electrodes 3 for collecting the parasitic signal, also makes it possible to measure the magnetic fields that are sources of interference very close to the location or locations where they act, which is advantageous for the processing for correction of the disrupted signals. The miniaturization of the housing 1′ makes it possible to consider as a variant the integration of the latter in the electrodes 3 themselves.

Thus, the invention relates to a process, a sensor device and an acquisition system comprising such a sensor device, making it possible to reduce and even to eliminate the artifacts that affect the electrophysiological signals and that are due to the magnetic fields.

Using the invention, it is therefore possible in particular to make a local measurement of fields and gradients of magnetic fields, which makes it possible to make a more exact correction of the interference linked to the magnetic field gradients, whereby these corrections can, for example, employ processing algorithms that are already known within the scope of prior art based on the knowledge of currents for generating magnetic field gradients.

Of course, the invention, which relates to a process, a device and a system for reducing the artifacts that affect the electrophysiological signals and that are due to electromagnetic fields, is not limited to the embodiments that are described and shown in the accompanying drawings. Modifications are possible, in particular from the standpoint of the composition of various elements or by substitution of equivalent techniques, without thereby exceeding the scope of protection of the invention.

1-23. (canceled)

24. Process for collecting—with a sensor device—at least one physiological signal from a living subject who is subjected to at least one electromagnetic field, in particular subjected to an electromagnetic environment such as the one existing in an investigative device that uses magnetic resonance, consisting in implementing a physical acquisition of at least one electrophysiological signal on the subject, and at least one pre-processing operation of said at least one electrophysiological signal that is detected; this is done via corresponding means,
process that is characterized in that it also consists in producing, using additional means (7, 8), a measurement of at least one characteristic of the electromagnetic environment at or in the immediate proximity of the point or points or zones (9) for acquisition of the electrophysiological signal(s) (SP) on the subject (2), and in correcting the—or each—electrophysiological signal (SP) that is detected and pre-processed, based on said at least one measuring signal (SM), optionally pre-processed, of at least one characteristic of the electromagnetic environment of the point or points or zones (9) for acquisition of the above-mentioned signal (SP), a characteristic that is selected from the group that is formed by the amplitude of the local magnetic field vector, in one or more directions, and the value of the magnetic field gradient(s) present, in one or more directions.

25. Process according to claim 24, wherein it also consists in carrying out, with suitable means (10, 11), a modulation, an adaptation to the transmission mode and an emission to a unit for control and remote processing (14), optionally combined with means for printing and/or display, said at least one pre-processed electrophysiological signal (SP) and said at least one measuring signal (SM) of at least one characteristic of the electromagnetic environment of the point or points or zones (9) for acquisition of the above-mentioned signal (SP), optionally also pre-processed, whereby said unit (14) also carries out the corrective processing of the signal (SP) based on the measuring signal (SM).

26. Process according to claim 24, wherein it also consists in carrying out the corrective processing of the—or of each—electrophysiological signal (SP) based on said at least one measuring signal (SM) using a suitable means (12) that is integrated with the sensor device (1) and then in carrying out, with suitable means (10, 11), a modulation, an adaptation to the transmission mode, and an emission to a unit for control and remote processing (14), optionally combined with means for printing and/or display of said at least one corrected electrophysiological signal (SP).

27. Sensor device of at least one physiological signal of a living subject who is subjected to at least one electromagnetic field, in particular subjected to an electromagnetic environment such as the one existing in an investigative device that uses magnetic resonance, comprising means for physical acquisition of at least one electrophysiological signal on the subject, such as electrodes, and at least pre-processing means of said at least one electrophysiological signal that is detected, a sensor device (1) that is characterized in that it also comprises additional means (7, 8) for the measurement of at least one characteristic of the electromagnetic environment at or in the proximity of the point or points or zones (9) for acquisition of the electrophysiological signal(s) (SP) on the subject (2), whereby said characteristic is selected from the group that is formed by the amplitude of the local magnetic field vector, in one or more directions, and the value of the magnetic field gradient(s) present, in one or more directions.

28. Sensor device according to claim 27, wherein the additional means comprise at least one magnetic field sensor (7) that provides a measuring signal (SM) that is based on the amplitude of the magnetic field vector at said sensor (7) and at least one means (8) for pre-processing the signal that is delivered by the—or each—sensor (7).

29. Sensor device according to claim 27, wherein it comprises several magnetic field sensors (7) that are used in combination, for example at least one pair of magnetic field sensors (7) that are arranged in a spaced manner in a determined direction.

30. Sensor device according to claim 27, wherein the additional means comprise at least one magnetic field sensor (7) in the form of a Hall effect sensor, or several magnetic field sensors (7) in the form of several Hall effect sensors that are used in combination, for example at least one pair of magnetic field sensors in the form of two Hall effect sensors (7) that are arranged in a spaced manner in a determined direction.

31. Sensor device according to claim 27, wherein it also comprises means (10, 11) for modulation, adaptation to the mode of transmission and emission for said at least one pre-processed electrophysiological signal (SP) and for said at least one measuring signal (SM) of at least one characteristic of the electromagnetic environment of the point or points or zones (9) for acquisition of the above-mentioned signal (SP), optionally also pre-processed.

32. Sensor device according to claim 27, wherein it also comprises, on the one hand, a means (12) for correction of the—or of each—electrophysiological signal (SP) that is detected and pre-processed based on said at least one measuring signal (SM), optionally pre-processed, of at least one characteristic of the electromagnetic environment of the point or points or zones (9) for acquisition of the above-mentioned signal (SP), and, on the other hand, a means (10, 11) for modulation, adaptation to the mode of transmission and emission for said at least one corrected electrophysiological signal (SP).

33. Sensor device according to claim 27, wherein the means (4; 5, 6) for pre-processing said at least one electrophysiological signal (SP) that is detected and the additional means (7, 8), as well as optionally the means (10, 11) for modulation and adaptation to the mode of emission and the means for correction (12), are produced in the form of integrated microelectronic components.

34. Sensor device according to claim 27, wherein it comprises several channels for acquisition and pre-processing of electrophysiological signals (SP) and several channels for measuring characteristics of the electromagnetic environment, making possible in particular the determination of the magnetic field gradients along the three axes of an orthogonal reference that is attached to said sensor device (1).

35. Sensor device according to claim 31, wherein the transmission of the electrophysiological signal(s) (SP) that are pre-processed or pre-processed and corrected, and, if necessary, the signal(s) (SM) for measuring the magnetic field, or magnetic field gradients, is carried out according to a serial transmission mode that is selected from the group that is formed by the transmission by optical fibers, the transmission by radiofrequency waves, and the transmission by shielded electrical conductors.

36. Sensor device according to claim 28, wherein each sensor (7) consists of a Hall sensor in the form of a microelectronic circuit of a standard CMOS semiconductor, preferably of the type that has a surface area of several mm² and a measurement precision on the order of 10 to 100 microtesla.

37. Sensor device according to claim 36, wherein the sensors (7) are integrated with the modules (4 and 8) for pre-processing the signals (SP and SM), as well as with circuits for multiplexing (MUX) and modulation (10), in the same microelectronic circuit (21) that is mounted in a housing (1′) that is connected to or that carries the electrodes (3), or else integrated in the latter.

38. Sensor device according to claim 36, further comprising a means (12) for correction of the—or of each—electrophysiological signal (SP) that is detected and pre-processed based on said at least one measuring signal (SM), optionally pre-processed, of at least one characteristic of the electromagnetic environment of the point or points or zones (9) for acquisition of the above-mentioned signal (SP), and, a means (10, 11) for modulation, adaptation to the mode of transmission and emission for said at least one corrected electrophysiological signal (SP), and wherein the circuit (21) also integrates the correction means (12).

39. System for acquisition of at least one electrophysiological signal of a living subject who is subjected to an MRI examination, whereby the system comprises in particular a unit for control and for processing and at least one sensor device that is connected to said unit, whereby said at least one sensor device is positioned on the subject, and the processing unit is located at a distance, wherein the—or each—sensor device (1) is a sensor device according to claim 27.

40. System according to claim 39, wherein the unit (14) for control and processing is connected to the—or to each—sensor device (1) by a bidirectional serial connection (15, 15′) that makes possible the transmission (15) of the—or of each—physiological signal (SP) that is collected by the sensor device(s) (1), and, if necessary, said at least one measuring signal (SM) of at least one characteristic of the magnetic environment at the measuring point(s) (9) of the sensor device (1) that is involved, to said unit (14) and the transmission (15′) of a clock signal from this unit (14) to said sensor device(s) (1), in particular for the purpose of modulation and multiplexing of signals that are collected by the—or each—sensor device (1).

41. System according to claim 39, wherein the unit (14) for control and processing comprises at least one means for editing the—or each—physiological signal (SP) that is collected, for example, a display means and/or a printing means.

42. System according to claim 39, wherein the unit for control and processing provides to the MRI device a sequencing signal that is extracted from the physiological signal (SP) that is collected.

43. System according to claim 40, wherein the unit (14) for control and processing comprises at least one means for editing the—or each—physiological signal (SP) that is collected, for example, a display means and/or a printing means.
